(19) United States
(12) Patent Application Publication
(10) Pub. No.: US 2006/0159645 A1
(43) Pub. Date: Jul. 20, 2006

(54) Method of providing lubricious surfaces

(76) Inventors: Jonathan Miller, Lawrenceville, NJ (US); Meghan Russell, Ewing, NJ (US); Lynn Halas, Ewing, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/238,815

(22) Filed: Sep. 29, 2005

Related U.S. Application Data
(60) Provisional application No. 60/615,844, filed on Oct. 4, 2004.

(51) Int. Cl. A61K 8/89

(52) U.S. Cl. 424/70.12; 424/401

(57) ABSTRACT

This invention relates to a method of providing lubricious surfaces, more particularly to a method of providing lubricious characteristics to skin surfaces that come into contact with other surfaces so as to prevent or treat chafing and/or irritation.

### FIELD OF THE INVENTION

This invention relates to a method of providing lubricious surfaces, more particularly to a method of providing lubricious characteristics to skin surfaces that come into contact with other surfaces. Such other surfaces include a second skin surface, clothing, including support hosiery, spandex, latex gloves, shoes or the like. Imparting lubricious characteristics to skin surfaces protects the skin surfaces from irritation, inflammation, chafing and may assist in preventing injury to the skin surfaces, thus avoiding violation or infection of the surfaces.

### BACKGROUND OF THE INVENTION

Human skin is an organ that surrounds and protects vital internal organs. Its integrity must be maintained or internal organs can become open to infection or damage. Any constant friction to which the skin is exposed can cause the skin to become irritated, chapped and, possibly, injured. This can be a particular problem among many individuals, whether caused by constant rubbing against another skin surface or by frictional exposure to an external surface such as cloth or solid surfaces including wood or metal, depending upon the individual's activity.

Individuals who participate in sports, such as running, rowing, or the like, may experience friction “burns”, itching, chapping or other irritation. Elderly patients especially those with circulatory problems may be prescribed support hosiery. The hosiery tends to fit very snuggly and may drag against the skin when removed at night or put on upon rising in the morning. Workers who constantly put on or remove other protective gear such as hand gloves may also experience friction type irritation. Likewise, skin surfaces of obese individuals often become chapped and sore due to the rubbing motion of their thighs against each other. Women with pendulous breasts may experience similar problems as their breasts rub against the abdominal skin underneath the breast surface. This is extremely painful and uncomfortable.

Heretofore, many individuals have treated or attempted to prevent such irritation or chapping by applying petroleum jelly, diaper ointment, powder, baby oil, skin lotion, personal lubricant, cream or some other like formulation to the appropriate skin surfaces. However, these preparations tend to be extremely messy as they remain on the surface of the skin without penetrating the skin. They, therefore, spread to clothing and stain it while becoming removed from the skin. Furthermore, particulate material such as powder may not afford the user protection from chafing or irritation. Once removed from the skin, they no longer provide the protection for which they were applied. Furthermore, they feel very greasy and uncomfortable to the user. Thus, there is a need for a method of treatment or prevention of chafing and irritation that affords protection to the user while remaining comfortable and aesthetically pleasing to the user.

### SUMMARY OF THE INVENTION

This invention relates to a method of providing protection to a first human skin surface that may be subject to frictional forces exerted by movement with respect a second surface by applying to the skin a composition comprising silicone gel or a topical composition containing the gel (e.g., a water-in-silicone composition) to said first skin surfaces. The compositions useful in the methods of this invention may also provide the additional benefit of being applied as an ointment or gel and drying to a powder-like consistency as a desirable smooth matte finish on the skin treatment area, thus providing an exceptionally comfortable feel to the user. The gel also provides the additional benefit of oil absorbing properties.

In one aspect, the method of this invention relates to the application of a silicone gel comprising a volatile liquid, a silicone polymer and a particulate ingredient to a first skin surface. In one embodiment, the silicone polymer is polysilicone-11. In one embodiment, the volatile liquid is a silicone fluid (e.g., cyclomethicone). In one embodiment, the silicone gel further comprises a second liquid (e.g., dimethicone). In one embodiment, the solvent is a non-alcohol solvent such as ester, (e.g., neopentyl glycol dioctanoate/diisostearate, octyl salicylate, and/or octyl methoxycinnimate and the like). In one embodiment, the silicone gel further comprises a particulate such as a porous silica (e.g., having a pore volume of 0.1 to about 1 ml/g, a particle diameter of between 1-20 microns, and/or an oil absorbance of between 10-500 ml/100 g) or other particulate matter such as talc, cornstarch or other particulate known to those of ordinary skill in the art.

In one embodiment, the gel further comprises petrolatum. In one embodiment, the gel further comprises additional dermatologically or therapeutically active agents such as benzoyl peroxide, resorcinol, sulfur, sodium borate, thymol, a retinoid, zinc sulfide, or zinc oxide, alpha-, beta-, or poly-hydroxy acids (e.g., lactic, glycolic, malic, tartaric, and citric acids and the like) and/or anti-microbial or anti-inflammatory agents (e.g., alpha-bisabolol, and the like). Zinc oxide may also be employed in the compositions useful in the methods of this invention. Anti-pruritics may also be useful in the methods of this invention.

In one embodiment of the method of this invention, the silicone gel to be applied to the first skin surface contains by weight: (a) from about 1% to about 99% (e.g., about 10% to about 80%) of the volatile liquid (e.g., cyclomethicone); (b) from about 1% to about 90% (e.g., about 10% to about 50%) of the silicone polymer (e.g., polysilicone-11); and (c) from about 0.001% to about 50% (e.g., about 1% and about 30%) of the salicylic acid. In a further embodiment, the silicone gel further comprises by weight: (d) about 0.001% to about 50% (e.g., about 0.001% to about 30%) of dimethicone; (e) about 0.001% to about 50% (e.g., about 0.001% to about 30%) of neopentyl glycol dioctanoate/diisostearate; (f) about 0.001% to about 50% (e.g., about 0.001% to about 30%) of porous silica; and/or (g) about 0.001% to about 20% (e.g., about 0.001% to about 5%) of alpha-bisabolol.

In another aspect, the method of this invention relates to applying to a first skin surface a composition containing: (a) the above described silicone gel; and (b) a cosmetically acceptable carrier to a first skin surface. In one embodiment, the cosmetically acceptable carrier contains one or more of the members selected from the group consisting of acidifying agents, alkalizing agents, aerosol propellants, antimicrobial agents, antioxidants, buffering agents, chelating agents, coloring additives, dermatologically active agents, dispersing agents, emollients, emulsifying agents, humectants, fragrances, preservatives, sugars, sunscreen agents, surfactants, suspending agents, thickening agents, and vehicles and the like.

In one embodiment, the composition is preferably a water-in-silicone emulsion comprising by weight: (a) about 0.001% to about 90% (e.g., about 1% to about 50%) of the silicone gel; (b) about 0.001% to about 50% (e.g., about 5% to about 50%). of liquid silicone (e.g., cyclomethicone, dimethicone, or mixtures thereof); and (c) water, e.g., q.s. to 100%, by weight, once all the other ingredients have been added. In a further embodiment, the composition further comprises by weight: (d) about 0.001% to about 50% (e.g., about 0.001% to about 20%) of a humectant (e.g., dipropylene glycol); and (e) about 0.001% to about 50% (e.g., about 0.001% to about 20%) of sunscreen (e.g., titanium dioxide).

In another aspect, the method of this invention relates to a method of treating or preventing chafing or irritation caused by friction of one skin surface against another skin surface. It also relates to a method of treating or preventing chafing or irritation caused by rubbing or friction of one skin surface against another surface, which may be a cloth surface or a hard surface. The method of this invention also relates to a method of promoting the treatment or prevention of chafing or irritation caused by friction of one skin surface against another surface such as skin, cloth or solid surface. A method of this invention relates to applying to the skin of a subject an effective amount of the above mentioned silicone gel or composition. In one embodiment, the gel or composition is applied to the skin one to three times daily. When used in treating chafing of one skin surface against another surface, the composition may be applied until the chafing or irritation is alleviated or it can be used prophylactically on a chronic basis. More preferably, the composition suseful in the methods of this invention are applied to two or more surfaces that may come into contact with each other.

The user may also, in accordance with the methods of this invention, apply such compositions to his or her skin which may come into frequent contact with external clothing or shoes, for example, in conjunction with athletic endeavors, in order to protect such skin from chafing, irritation and/or blistering should said skin rub against clothing.

Another method of treating or preventing chafing or irritation in accordance with this invention relates to the employment of the compositions set forth herein to treat diaper rash, athlete's foot or other condition caused by microorganisms such as Candida albicans in locations in which skin surfaces are rubbed together and may create humid or moist conditions. Preferably, the composition useful in preventing or treating diaper rash contain an antifungal and/or antibacterial active ingredient, such as an imidazoles antifungal or the like. The method of treating diaper rash involved applying at each diaper change compositions of this invention to the perineal surfaces prior to applying a diaper. This method may be used in conjunction with infants, children and/or adults.

In one aspect, the method of this invention features the application to one or more skin surfaces of a silicone gel comprising a volatile liquid, a silicone polymer, and a particulate such as silica. In one embodiment, the silicone polymer is polysilicone-11. In one embodiment, the volatile liquid is a silicone fluid (e.g., cyclomethicone). In one embodiment, the silicone gel further comprises a second liquid (e.g., dimethicone). In one embodiment, the solvent is a non-alcohol solvent such as ester, e.g., neopentyl glycol dioctanoate/diisostearate, octyl salicylate, and octyl methoxycinnamate). In yet another embodiment, the silicone gel further comprises a porous silica (e.g., having a pore volume of 0.1 to about 1 ml/g, a particle diameter of between 1-20 microns, and/or an oil absorbance of between 10-500 ml/100 g). In one embodiment, the gel further comprises petrolatum. In one embodiment, the gel further comprises additional dermatologically active agents such as the anti-acne agents (e.g., benzoyl peroxide, resorcinol, sulfur, sodium borate, thymol, a retinoid, zinc sulfide, or zinc oxide), alpha.-, beta.-, and poly-hydroxy acids (e.g., lactic, glycolic, malic, tartaric,.and citric acids and the like) and/or anti-microbial or anti-inflammatory agents.(e.g., alpha-bisabolol).

In one embodiment, the silicone gel comprises by weight: (a) about 1% to about 99% (e.g., about 10% to about 80%) of the volatile liquid (e.g., cyclomethicone); (b) about 1% to about 90% (e.g., about 10% to about 50%) of the silicone polymer (e.g., polysilicone-11); and (c) about 0.001% to about 50% (e.g., about 1% and about 30%) of the salicylic acid. In a further embodiment, the silicone gel further comprises by weight: (d) about 0.001% to about 50% (e.g., about 0.001% to about 30%) of dimethicone; (e) about 0.001% to about 50% (e.g., about 0.001% to about 30%) of neopentyl glycol dioctanoate/diisostearate; (f) about 0.001% to about 50% (e.g., about 0.001% to about 30%) of porous silica; and/or (g) about 0.001% to about 20% (e.g., about 0.001% to about 5%) of alpha-bisabolol.

In another aspect, the methods of this invention feature application to one or more skin surfaces a composition comprising: (a) the above described silicone gel; and (b) a cosmetically acceptable carrier. In one embodiment, the cosmetically acceptable carrier comprises one or more of the members selected from the group consisting of acidifying agents, alkalizing agents, aerosol propellants, antimicrobial agents, antioxidants, buffering agents, chelating agents, coloring additives, dermatologically active agents, dispersing agents, emollients, emulsifying agents, humectants, fragrances, preservatives, sugars, sunscreen agents, surfactants, suspending agents, thickening agents, and vehicles.

The methods of this invention may also be utilized in conjunction with application of medical support or elastic garments, as well as in conjunction with medical or other professionals who need to remove and replace garments such as gloves multiple times. In such cases, the compositions set forth herein may be applied to the skin surface prior to putting on the garment. Preferably, the compositions of this invention may be applied to both the skin surface and the interior of the garment which will contact the skin surface.

In yet another embodiment, the compositions useful in the methods of this invention are water-in-silicone emulsions comprising by weight: (a) about 0.001% to about 90% (e.g., about 1% to about 50%) of the silicone gel; (b), about 0.001% to about 50% (e.g., about 5% to about 50%) of liquid silicone (e.g., cyclomethicone, dimethicone, or mixtures thereof); and (c) water, e.g., q.s. to 100%, by weight, once all the other ingredients have been added. In a further embodiment, the composition further comprises by weight: (d) about 0.001% to about 50% (e.g., about 0.001% to about 20%) of a humectant (e.g., dipropylene glycol); and (e) about 0.001% to about 50% (e.g., about 0.001% to about 20%) of sunscreen (e.g., titanium dioxide).

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

The method of this invention relates to applying to a first skin surface a silicone gel composition that both delivers skin protection properties while simultaneously possessing aesthetically pleasing properties to the skin. The present invention also features a cosmetic composition containing the gel (e.g., the gel is dispersed throughout the composition). The silicone gel is made, in one embodiment, by dispersing oil swellable silicone polymer (e.g., polysilicone-11) in a volatile liquid (e.g., silicone fluid such as cyclomethicone) or a volatile liquid mixed with other ingredients (e.g., dimethicone).

Upon application to the skin, the silicone gel releases the volatile liquid, and delivers to the skin a “powdery”, feel that is pleasing to the user and yet continues to deliver the benefit of lubrication and slip between the skin surface and other surfaces such as other skin surfaces or external clothing. The silicone gel may also contain porous silica that further we believe enhances the aesthetic and lubricating properties of the gel. We believe that this powder feel comes about as a result of the release of volatile ingredients in the compositions of this invention upon application to the skin or other surface. The gel additionally has the ability to scatter incoming rays of light in all directions. By diminishing reflected light, the skin, thus, has a smooth matte appearance and feel.

As used herein, the term “volatile” refers to those liquids that have a measurable vapor pressure at ambient temperature. Examples of volatile liquids include branched or straight chained hydrocarbons (e.g., C3-C20 hydrocarbons such as isoparaffins, isoeicosane, isohexadecane and isododecane) and silicone fluids. Examples of volatile silicone fluids include the following: cyclic and linear polydimethylsiloxanes containing from about 3 to about 9 (e.g., from about 4 to about 5) silicone atoms such as cyclomethicones; Dow Corning 200, Dow Coring 344, and Dow Corning 345 (manufactured by Dow Corning, Midland, Mich.); Silicone 7158 and 7207 (manufactured by Union Carbide, Houston, Tex.); SF 1202 (manufactured by General Electric); and SWS-03314 (manufactured by SWS Silicones, Inc.). As used herein, the term cyclomethicone refers to cyclotrisiloxane, cyclotetrasiloxane, cyclopentasiloxane, cyclohexasilxane, or mixtures thereof.

The silicone polymers useful in the methods of the present invention may have an average molecular weight in excess of 10,000 (e.g., between about 10,000 and 10,000,000). Examples of silicone polymers include crosslinked siloxane (e.g., dimethicone or dimethicone derivatives) copolymers such as stearyl methyl-dimethyl siloxane copolymer (Gransil SR-CYC, available from Grant Industries, Elmwood Park, N.J.); dimethicone/vinyldimethicone crosspolymers; Polysilicone-11 (i.e., a crosslinked silicone rubber formed by the reaction of vinyl terminated silicone and methylhydrodimethyl siloxane in the presence of cyclomethicone), cetearyl dimethicone/vinyl dimethicone crosspolymer (i.e., a copolymer of cetearyl dimethicone crosslinked wit vinyl dimethyl polysiloxane), dimethicone/phenyl vinyl dimethicone crosspolymer (i.e., copolymer of dimethylpolysiloxane crosslinked with phenyl vinyl dimethylsiloxane), and dimethicone/vinyl dimethicone crosspolymer (i.e., copolymer of dimethylpolysiloxane crosslinked with vinyl dimethylsiloxane). More preferably, the compositions useful in the, method of this invention include silicone elastomer blands containing dimethicone/vinyldimethicone crosspolymers (such as those made by Dow Corning), dimethicone, cyclopentailoxane, trisiloxane, dimethicone and silica. More preferably, the compositions useful in the methods of this invention relate to non-aqueous or anhydrous compositions.

Silicone gels may also be purchased from commercial suppliers such as Grant Industries. Examples of such gels include cyclomethicone (and) polysilicone-11 (Gransil GCM5), cyclotetrasiloxane(D4) (and) petrolatum (and) polysilicone-11 (Grangil PS-4), cyclopentasiloxane(D5) (and) petrolatum (and) polysilicone-11 (Gransil PS-5), cyclopentasiloxane(D5) (and) dimethicone (and) polysilicone-11 (Gransil DMCM-5), cyclotetrasiloxane(D4) (and) dimethicone (and) polysilicone-11 (Gransil DMCM-4), polysilicone-11 (and) isododecane (Gransil IDS), and cyclomethicone (and) polysilicone-11 (and) petrolatum (and) phytosphingosine (Gransil SPH). Examples of such gels available from General Electric include cyclopentasiloxane (and) dimethicone/vinyl dimethicone crossploymer (SFE839). In general, the compositions set forth in U.S. Pat. No. 6,200,964 and U.S. Pat. No. 6,384,023, which are hereby incorporated herein by reference, are suitable for use in the methods of this invention.

The invention features a method of applying a cosmetic composition suitable for application to the skin, e.g., under the breasts or on the thighs, of a subject in association with a cosmetically acceptable carrier. The individual components of the carrier are numerous and varied, but are also well known to one skilled in the art. In one aspect, the carrier comprises one or more of the members selected from the group consisting of acidifying agents, alkalizing agents, aerosol propellants, antimicrobial agents, antioxidants, buffering agents, chelating agents, coloring additives, dermatologically active agents, dispersing agents, emollients, emulsifying agents, humectants, fragrances, masking agents, preservatives, sugars, sunscreen agents, surfactants, suspending agents, thickening agents, an vehicles. These ingredients are discussed below. Examples of these agents are listed below as well as in the International Cosmetic Ingredient Dictionary and Handbook, eds. Wenninger and McEwen (The Cosmetic, Toiletry, and Fragrance Assoc., Washington, D.C., 7.sup.th Edition, 1997) (hereinafter “ICT Handbook”).

Acidifying and alkalizing agents are preferably added to obtain the desired pH of the composition. Examples of acidifying agents included acetic acid, citric acid, glacial acetic acid, malic acid, and proprionic acid. Examples of alkalizing agent include edetol, potassium carbonate, potassium hydroxide, sodium borate, sodium carbonate, and sodium hydroxide. Other acidifying and alkalizing agents are listed on page 1653 of the ICT Handbook.

Aerosol propellants are used when the composition is to be administered as an aerosol under pressure. Examples of aerosol propellants include halogenated hydrocarbons such as dichlorodifluoromethane, dichlorotetrafluoroethane, and trichloromonofluoromethane, nitrogen, and volatile hydrocarbons such as butane, propane, isobutane, or mixtures thereof. Other propellants are listed on page 1655 of the ICT Handbook.

Anti-microbial agents are used when the area that the composition is to be applied is prone to microbial infection, e.g., by bacteria, fungal, or protozoa. Examples of such agents include benzyl alcohol, chlorobutanol, phenylethyl alcohol, phenylmercuric acetate, potassium sorbate, and sorbic acid, benzoic acid, butyl paraben, ethyl paraben, methyl paraben, propyl pareben, and sodium benzoate. Other anti-microbial agents are listed on page 1612 of the ICT Handbook.

Antioxidants are used to protect ingredients of the composition from oxidizing agents that are included within or come in contact with the composition. Examples of antioxidants include water soluble antioxidants such as ascorbic acid, sodium sulfite, metabisulfite, sodium bisulfite, sodium formaldehyde, sulfoxylate, isoascorbic acid, isoascorbic acid, cysteine hydrochloride, 1,4-diazobicyclo-(2,2,2)-octane, and mixtures thereof. Examples of oil-soluble antioxidants include ascorbyl palmitate, butytlated hydroxyanisole, butylated hydroxytoluene, potassium propyl gallate, octyl gallate, dodecyl gallate, phenyl-.alpha.-napthyl-amine, and tocopherols such as alpha.-tocopherol. Other antioxidants are listed on pages 1612-13 of the ICT Handbook.

Buffering agents are used to maintain an established pH of the composition. Examples of buffering agents included sodium citrate, calcium acetate, potassium metaphosphate, potassium phosphate monobasic, and tartaric acid. Other buffering agents are listed on page 1653 of the ICT Handbook.

Chelating agents are used to maintain the ionic strength of the composition and/or bind to destructive compounds and metals that are included within or come in contact with the composition. Examples of chelating agents included dihydroxy ethyl glycine, citric acid, tartaric acid, edatate dipotassium, edetate disodium, edetic acid, and ethylenediamine tetracetic acid (EDTA) and its salts (e.g., tetrasodium EDTA). Other chelating agents are listed on page 1626 of the ICT Handbook.

Coloring additives are used to add color to the composition. Examples of such coloring additives include titanium dioxide, yellow iron oxide, red iron oxide, black iron oxide, caramel, carmine, fluorescein derivatives, methoxsalen, trioxsalen, carbon black, azo dyes, anthraquinone dyes, blue azulenes, guajazulene, chamuzulene, erythrosin, bengal rose, phloxin, cyanosin, daphinin, eosin G, cosin 10B, and Acid Red 51. Other coloring agents are listed on pages 1628-30 of the ICT Handbook.

Dermatologically active agents include agents for treating wound healing, inflammation, acne, psoriasis, cutaneous aging, skin cancer, impetigo, herpes, chickenpox, dermatitis, pain, itching, and skin irritation. Examples of such dermatologically active agents include hydrocortisone, dexamethasone, panthenol, phenol, tetracycline hydrochloride, yeast, hexylresorcinol, lamin, kinetin, betamethagone, triamcinolone, fluocinolone, methylprednisolone, retinoids such as retinol and retinoic acid, dapsone, sulfasalazine, resorcinol, salicylic acid, benzoyl peroxide, erythromycin-benzoyl peroxide, erythromycin, clindamycin, mupirocin, griseofulvin, azoles such as miconazole, econazole, itraconazole, fluconazole, and ketoconazole, ciclopirox, allylamines such as naftifine and terfinafine, acyclovir, famciclovir, valacyclovir, benzocaine, lidocaine, dibucaine, pramoxine hydrochloride, methyl salicylate, camphor, menthol, resocinol, and vitamins such as tocopherol, tocopheryl acetate, pentothenic acid, ascorbic acid, biotin, and retinoids such as retinol, retinoic acid, retinal, retinyl acetate, and retinyl palmitate, .alpha.-hydroxy acid, a .beta.-hydroxy acid, or poly-hydroxy acid such as glycolic acid, lactic acid, citric acid, malic acid, and azaleic acid, and sunless tanning agents such as 1,3-dihydroxyacetone and 1,3,4-trihydroxy-2-butanone (erythulose).

Examples of dispersing and suspending agents include quarternium-18 hectorite, polyhydroxy stearic acid, poligeenan and silicon dioxide. Other dispersing and suspending agents are listed on page 1690-91 of the ICT Handbook.

Emollients are agents that soften and smooth the skin. Examples of emollients include hydrocarbon oils and waxes (e.g., natural and synthetic waxes) such as mineral oil, petrolatum, microcrystaline wax, polyethylene, triglyceride esters such as those of castor oil, cocoa butter, safflower oil, cottonseed oil, corn oil, olive oil, cod liver oil, almond oil, avocado oil, palm oil, sesame oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholidds; and sterols. Other emollients are listed on pages 1656-61 of the ICT Handbook.

Emulsifying agents are used for preparing emulsions of the present invention. Examples of emulsifying agents used for preparing water-in-oil emulsions include cyclomethicone (and) dimethicone copolyol, dimethicone copolyol, cetyl dimethicone copolyol, PEG-30 dipolyhydroxystearate, and PEG-40 sorbitan peroleate. Examples of emulsifying agents used for preparing oil-in-water emulsions of the present invention include glyceryl stearate, PEG-100 stearate, methyl gluceth sesquisterate, fatty alcohols, and alkyl phenols condensed with ethylene oxide. Other emulsifiers are listed on pages 1679-87 of the ICT Handbook. Emulsion stabilizers are listed on pages 1634-35 of the ICT Handbook.

Humectants are agents that promote the retention of moisture, e.g., moisturizers. Examples of humectants include sorbitol, matricaria extract, aloe barbadensis gel, glycerin, glycereth 5 lactate, glycereth 7 triacetate, glycereth 7 diisononoate, hexanetriol, hexylene glycol, propylene glycol, dipropylene glycol, alkoxylated glucose, D-panthenol, 1-2-pantandiol, 2-methyl-1,3-propanediol, and derivatives thereof, and hyaluronic acid. Other humectants are listed on pages 1661-62 of the ICT Handbook.

Examples of fragrances include peppermint, rose oil, rose water, aloe vera, clove oil, menthol, camphor, eucalyptus oil, and other plant extracts. Certain fragrances may require a solubilizer, e.g., PPG-5-ceteareth-20. To eliminate certain odors from compositions, masking agents may be used. An example of a masking agent includes ethylene brassylate. Other fragrances and masking agents are listed on pages 1639-40 of the ICT Handbook.

Preservatives are used to protect the composition from degradation. Examples of preservatives include liquipar oil, phenoxyethanol, methyl paraben, propyl paraben, butyl paraben, isopropyl paraben, isobutyl paraben, dieizolidinyl urea, imidazolidinyl urea, diazolindyl urea, benzalkonium chloride, benzethonium chloride, phenol, and mixtures thereof (e.g., liquipar oil). Other preservatives are listed on pages 1654-55 of the ICT Handbook.

Examples of sugars include monosaccharides, disaccharides, and polysccharides such as glucose, xylose, fructose, reose, ribose, pentose, arabinose, allose, tallose, altrose, mannose, galactose, lactose, sucrose, erythrose, glyceraldehyde, or any combination thereof.

Sunscreen agents are agents used to block or reduce the amount of ultraviolet radiation impinging on the skin (e.g., by absorption, scattering, and reflection of the ultraviolet radiation). Segarin, et al., Cosmetics Science and Technology, Chapter VIII, pages 189, et seq. discloses numerous examples of sunscreen agents. Examples of sunscreen agents include both organic compounds and their salts such as octyl methoxycinnamate, octyl salicylate, benzophenone-3 homosalate, octocrylate, avobenzone, and menthyl anthranilate, as well as inorganic particulate materials such as zinc oxide, silica, iron oxide, titanium dioxide, and 2-ethyl-hexyl-p-methoxycinnamate. Other sunscreen agents are listed on page 1672 of the ICT Handbook. Generally, the composition will contain from about 1% to about 30%, by weight, of sunscreen agent(s). The exact amounts will vary depending on the sunscreen used and the desired sun-protection factor (SPF).

Surfactants are agents used to stabilize multi-component compositions, e.g., used as wetting agents, antifoam agents, emulsifiers, dispersing agents, and penetrants. Examples of surfactants include methyl gluceth 20, decyl polyglucoside, lapyrium chloride, laureth 4, laureth 9, monoethanolamine, nonoxynol 4, nonoxynol 9, nonoxynol 10, nonoxynol 15, nonoxynol 30, poloxalene, polyoxyl 8, 40, and 50 stearate, polysorbate 20, polysorbate 40, polysorbate 60, polysorbate 65, polysorbate 80, and polysorbate 85, sodium lauryl sulfate, sorbitan and its derivatives. Other surfactants are listed on page 1672-90 of the ICT Handbook.

Vehicles are often referred to as the base for the cosmetically acceptable carrier, e.g., a fluid that is capable of delivering the other components of the composition to the skin with acceptable absorption of those components into the skin. Examples of vehicles include water, e.g., deionized water, saline (e.g., sodium chloride dissolved in deionized water), oil-in-water emulsions (e.g., where the Continuous water phase contains the water soluble agents and the discontinuous oil phase contains the oil soluble agents), and water-in-oil emulsions (e.g., where the continuous oil phase contains the oil soluble agents and the discontinuous water phase contains the water soluble agents). The oil phase may be established by the addition of hydrocarbon and/or silicone fluids, e.g., cyclomethicone and dimethicone, together with various suitable emulsifying agents. In order to reduce applying additional oil to the skin of the subject, hydrocarbon oils should be avoided.

The cosmetically acceptable carrier that may be in a number of different delivery forms, e.g., a spray, mist, aerosol, shampoo, hair conditioner, mousse, semi-solid cream, liquid such as a solution, emulsion, or suspension, lotion, gel, solid such as a powder, adherent stick, flexible mask, self-hardening liquid or gel, or other suitable forms intended to be applied to the skin of a subject (e.g., a human). Water-in-oil emulsions (e.g., ratio of about 2:1 to about 1:100 such as about 1:1 to about 1:10) and oil-in-water emulsions (e.g., ratio of about 2:1 to about 1:100 such as about 1:1 to about 1:10) are typically used in preparing lotions and creams. The actual ratio of the two phases will depend on the consistency of the desired final product.

The viscosity of the compositions of the present invention may be different dependent upon the type of formulation being prepared, e.g., a liquid formulation will have a lower viscosity than a gel or cream formulation. Typically, the viscosity of liquid formulations of the present invention will range from 5,000 to 25,000 cps. Bulking agents may be used to increase the viscosity of the composition. An example of a bulking agent is talc. Other bulking agents are listed on page 1625-26 of the ICT Handbook. Other viscosity increasing agents are listed on pages 1693-97 of the ICT Handbook. Viscosity decreasing agents are listed on pages 1692-92 of the ICT Handbook.

The compositions useful in the methods of this invention may be prepared using methodology that is well known by an artisan of ordinary skill (e.g., by using well-known mixing and blending procedures). For examples, for emulsion compositions of the present invention, each phase of the emulsion may be separately prepared with all of the components contained in their appropriate phases. The emulsion is then formed by adding one phase to the other phase with agitation.

The gel or ointment compositions useful in the methods of this invention may be packaged in a container that is well known by an artisan of ordinary skill, e.g., the silicone gel may be packaged in a low density polyethylene tube with a dispensing tip head and the cosmetic foundation of the present invention may be packaged in a glass or plastic bottle.

The following is a description of the manufacture of specific gels and compositions of the present invention. Other gels and compositions of the invention can be prepared in an analogous manner by a person of ordinary skill in the art.

### EXAMPLE 1

Topical Silicone Gel

The ingredients and their respective weight percentages with respect to the total composition for the cosmetic product of Example 1 are recited below in Table 1.

TABLE 1

| INGREDIENT | WEIGHT (%) |
|---|---|
| Cyclopentasiloxane(D5) (and) Dimethicone | 85.81 |
| (and) Polysilicone-11 (30:30:40) | |
| Neopentyl Glycol Dioactonate/Diisostearate | 5.50 |
| Alpha-bisabolol | 0.10 |
| Fragrance | 0.04 |
| Porous Silica | 8.55 |
| | 100.00 |

The cyclopentasiloxane (D5) (and) dimethicone (and) polysilicone-1l gel (Gransil DMCM-5, Grant Industries) may be added to a main beaker. The neopentyl glycol dioctnoate/diisosteaate (Minno 21, Bernel Co., Elmwood, N.J.) may then be added to a second beaker, mixed, and heated to between 500 to about 60° C. The solution may be allowed to cool to room temperature. Alpha-bisabolol and the fragrance may then be added to the solution in the second beaker and mixed until homogenous. The ingredients of the second beaker may then be added to the main beaker and mixed until homogenous. Lastly, silica may be slowly added to the main beaker while mixing until uniform. The resulting gel should have a viscosity of between 200,00 and 400,000 cps.

### EXAMPLE 2

Water-in-silicone Cosmetic Skin Composition Containing Silicone GelB

The ingredients and their respective weight percentages with respect to the total composition for the cosmetic product of Example 2 are recited below in Table 2.

TABLE 2

| INGREDIENT | WEIGHT (%) |
|---|---|
| Phase A | |
| Deionized Water | 32.95 |
| Sodium Chloride | 0.50 |
| Matricaria Extract | 1.00 |
| Aloe Barbadensis Gel | 0.01 |
| Diazolindyl Urea | 0.20 |
| Phase B | |
| Dipropylene Glycol | 4.00 |
| Methyl paraben | 0.15 |
| Phase C | |
| Polyglyceryl-4 Oleate (and) PEG-8 Propylene | 2.00 |
| Glycol Cocoate (80:20) | |
| Cyclomethicone (and) Dimethicone Copolyol | 13.00 |
| (90:10) | |
| Propyl paraben | 0.20 |
| Cyclomethicone (and) Quarternium18 (and) | 1.50 |
| Hectorite (and) Propylene Carbonate (35:60:5) | |
| Ethylene Brassylate | 0.20 |
| Synthetic Wax | 1.20 |
| C12-15 Alkyl Benzoate (and) Titanium Dioxide | 8.00 |
| (and) Alumina (and) Polyhydroxystearic Acid | |
| (and) Silica (60:30:5:2.5:2.5) | |
| Phase D | |
| Cyclomethicone | 9.69 |
| Phase E | |
| Talc | 6.40 |
| Silica Silyate | 0.50 |
| Polymethyl Methacrylate (and) tocopheryl | 0.10 |
| acetate (and) pantothenic acid (and) ascorbic | |
| acid (and) retinyl palmitate (90:7:1:1:1) | |
| Titanium Dioxide | 7.71 |
| Yellow Iron Oxide | 0.49 |
| Red Iron Oxide | 0.15 |
| Black Iron Oxide | 0.05 |
| Phase F | |
| Cyclomethicone (and) | 10.00 |
| Polysilicone-11 (87.5:12.5) | |
| | 100.00 |

The suppliers of the above listed ingredients may be the following: matricaria extract (Active Oraganics; Lewisville, Tex.), polyglyceryl-4 oleate (and) PEG-8 Propylene Cocoate (Henkel, Dusseldorf, Germany); cyclomethicone (and) dimethicone copolyol (Dow Corning, Midland, Mich.); cyclomethicone (and) quarternium-18 (and) hectorite (and) propylene carbonate (Rheox, Philadelphia, Pa.); synthetic wax (Presperse, Piscataway, N.J.); C12-15 alkyl benzoate (and) titanium dioxide (and) polyhydroxystearic acid.(and) silica (Tioveil, Durham, England); polymethyl methacrylate (and) tocopheryl acetate (and) pantothenic acid (and) ascorbic acid (and) retinyl palmitate (Brooks Industries; South Plainfield, N.J.); iron oxides (U.S. Cosmetic Corporation, Dayville, Conn.); and Cyclomethicone (and) polysilicone-11 (Gransil GCM5; Grant Industries, Elmwood Park, N.J.). The Cyclomethicone (and) polysilicone-11 (and) salicylic acid may be manufactured by adding solid salicylic acid powder to the silicone gel and milling the mixture to ensure homogeneous distribution of the salicylic acid in the silicone gel. The cyclomethicone that may be used in the silicone gel may be cyclopentasiloxane, but other cyclomethicones may be used (e.g., cyclotetrasiloxane).

The Phase C ingredients may be added, in the order listed, into a stainless steel jacketed oil-phase kettle equipped with variable speed propeller agitation. The agitation should be begun at a temperature of 20-30 degree C. as soon as the propeller is covered sufficiently to mix the ingredients without splashing (“Phase C Mixture”). Cold water should be used in the jacket as necessary to remove any heat generated from mixing and milling.

A W750 Colloid Mill (Greerco/Chemineer, North Andover, Mass.) may be connected to the oil-phase kettle containing the Phase C Mixture and was set up to recirculate. A drop-in homogenizer may also be placed in the oil-phase kettle. The propeller mixer in the oil-phase kettle may be adjusted to create a vortex in the Phase C Mixture, and the ingredients of Phase E are added. After all the pigments have been added, the propeller speed may be reduced so that air was not whipped into the mixture. The homogenizer and the colloid mill may then be turned on. The gap set of the mill should be initially set at “40,” but then immediately closed to between “4”-“6” (“Phase CE Mixture”).

50-80% of the cyclbmethicone in Phase D may be added to the Phase CE Mixture to adjust the viscosity of the slurry so that it is a suitable viscosity to colloid mill properly. The batch may then be homogenized and milled (e.g., for at least 2 hours) until the dispersion is free of color spots when checked between two glass slides (“Phase CED Mixture”).

The Gransil CGM5-SA of Phase F may then be added to the Phase CED Mixture. Milling should be continued for 20 minutes to insure that the Gransil CGM %-SA is uniformly dispersed. The gap set of the mill may be opened to 40, and the colloid mill then turned off. The colloid mill should be used to transfer the mixture to the main kettle. All of the remaining cyclomethicone from Phase D may be used to rinse the bottom of the oil-phase kettle and the mill and added to the main kettle. The mill may then be attached to the main kettle and set up to recirculate (“Phase CEDF Mixture”).

The Phase A ingredients may then be added, in the order listed, into a stainless steel jacketed water-phase kettle equipped with variable speed propeller agitation. The agitation should be begun as soon as the propeller is covered sufficiently to mix without splashing (“Phase A Mixture”). The Phase B dipropylene glycol should then be placed into a suitable vessel, and the methyl paraben of Phase B added the vessel and mixed until completely dissolved (“Phase B Mixture”). The Phase B Mixture should be added to the water-phase kettle, and the resulting mixture mixed with medium propeller agitation for a minimum of ten minutes or until clear (“Phase AB Mixture”).

The Phase AB Mixture in the oil-phase kettle should then be added to the Silicone/Color Phase in the main kettle, i.e., Phase CEDF Mixture, in the following manner. A pump should be connected to the water-phase kettle. The flow rate of the pump should be checked by measuring the weight and time it takes to fill a 5-gallon pail about ½-¾ full. The Phase AB Mixture addition rate should be between 2 and 4 kg per minute. The Phase AB Mixture should then be added at a rate so that water did not pool on the surface of the main kettle. If water began to pool on the surface, the transfer pump should be shut off, and the pooled water allowed to be incorporated before continuing. The sweep and propeller mixers should be activated during the water phase addition. As the level of the batch rose, the speed of the mixers should be adjusted to maintain good mixing without splashing.

After the water phase addition is complete, the colloid mill (gap open at “40”) should be run for 15 seconds to flush out the bottom of the tank. The propeller and sweep mixers should be allowed to mix the batch for 15 minutes. Upon completion of matching the shade and color of the composition, the viscosity of the batch should be set by using the colloid mill to impart high sheer on the mixture in a single pass as it is removed from the main kettle. The gap on the colloid mill should be set between “4” and “6” and the main kettle drained.

The resulting composition should have a viscosity of 14,000 cps. Other shades of this foundation can be obtained by varying the ratios of the iron oxide colorants.

### EXAMPLE 3

Another embodiment of the compositions useful in the methods of this invention include the following:

| INGREDIENT | WEIGHT (%) |
|---|---|
| Dimethicone/Vinyldimethicone Crosspolymer | 20-23% |
| Dimethicone (5 and 350 cst) | 18-22% |
| Cyclopentasiloxane | 35-37% |
| Trisiloxane | 5-6% |
| Decamethyltetrasiloxane | 6-7% |
| Dodecamethylpentasiloxane | 3-45% |
| Silica | 3.5-4% |
| Antioxidant | 0.1% |
| Dimethicone, NF | 1-2% |

The composition of this example may be made in accordance with the method set forth in Example 1.

### EXAMPLE 4

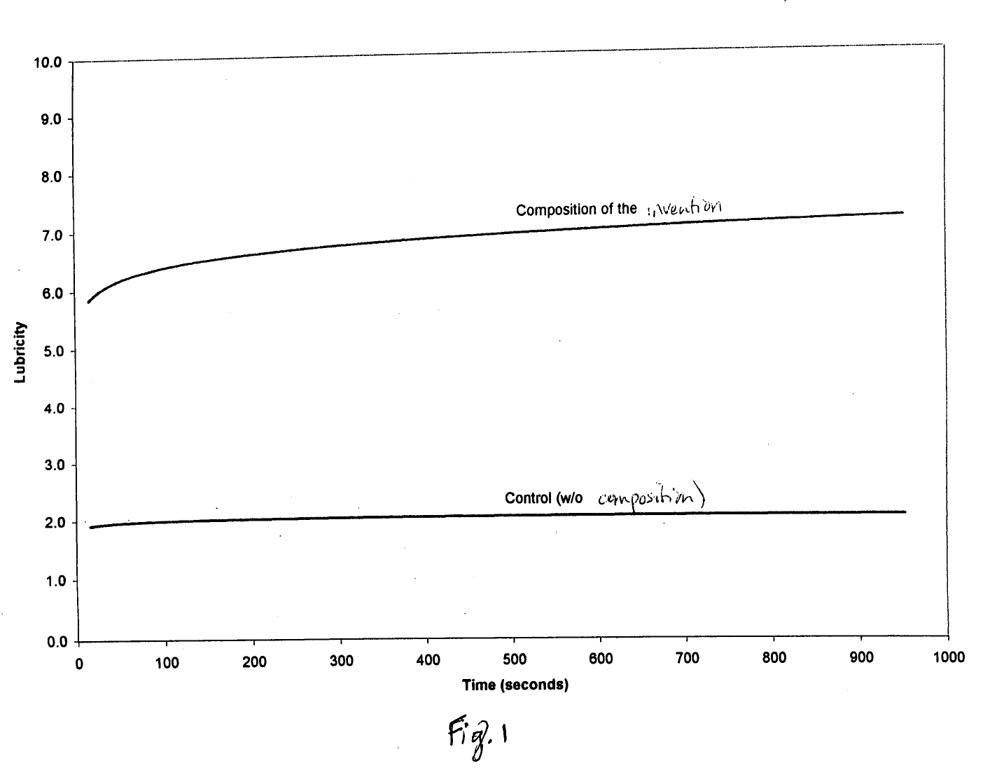

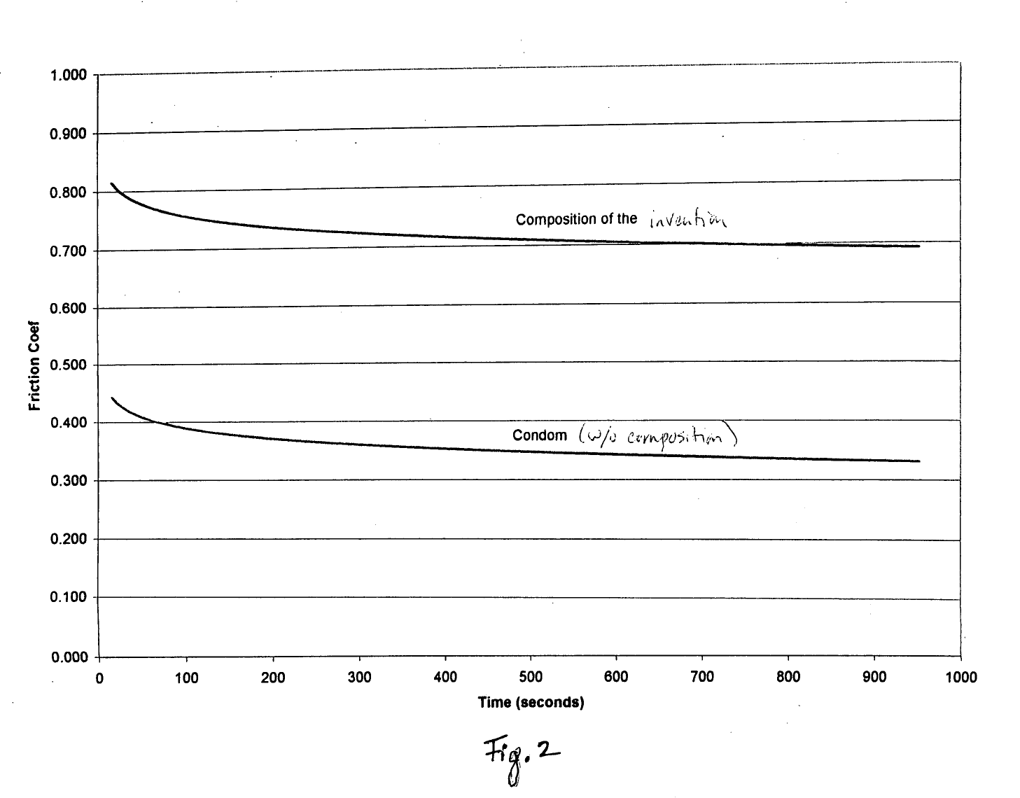

Ahmad et al. in U.S. Pat. No. 6,139,848, which is hereby incorporated herein by reference, describe a method to test lubricity of various personal lubricants known to the trade. In the described test method, the lubricity of various marketed personal lubricants was determined over a period of 300 seconds (5 minutes). The test set forth in U.S. Pat. No. 6,139,848 was modified herein by rubbing the composition of Example 3 above on both test surfaces. The lubricity of the composition of Example 3 was compared with lubricity of a condom without coating. The resulting lubricity data are set forth in FIGS. 1 and 2 herein. FIG. 1 shows the relative lubricity of a condom that has not been coated with said composition compared with the lubricity of two surfaces, both coated with said composition. FIG. 2 shows the relative coefficient of friction of a condom that has not been coated with said composition compared with the lubricity of two surfaces, both coated with said composition. These data indicate that the lubricity of the composition of Example 3 when rubbed onto both test surfaces is about twice that of an uncoated condom. This demonstrates the ability of the methods of this invention to prevent or treat chafing or irritation when compositions are applied to surfaces that contact one another.

### EXAMPLE 5

The methods of this invention were used by a group of 157 women between the ages of 25 and 54. They applied the composition of Example 3 to areas of their body in which they experienced chafing and irritation, including their thighs and under their breasts. They reported that the composition was not irritating, was gentle enough for everyday use, allowed their skin to breathe and was safe and gentle enough for use in intimate areas. They also found that the methods of this invention provided relief to areas that experienced chafing. They found that their skin was soothed and cooled, their chafing soother, that the method provided long-lasting relief, relieved irritation from chafing, reduced the appearance of chafing, gave relief from rubbing and that relief started after just one application. Furthermore, the methods of this invention were found to protect the skin surfaces to which they were applied, including the following attributes: provided a silky barrier, prevented chafing, protected skin from friction and irritation, guarded skin against rashes caused by wetness and friction, fought friction, protected skin against wetness and formed a breathable barrier. They also found it very easy to apply.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A method of treating or preventing chafing of a skin surface comprising applying to a skin surface a composition comprising silicone and a particulate.

2. A method according to claim 1 wherein said composition is a silicone-containing composition comprising at least one volatile liquid., a silicone polymer and a particulate ingredient.

3. A method according to claim 2 wherein said particulate is selected from the group consisting of silica, talc and cornstarch.

4. A method according to claim 1 wherein said method further comprises applying said composition to a second surface that is expected to come into contact with said skin surface.

5. A method according to claim 4 wherein said second surface is a skin surface.

6. A method of treating diaper rash comprising applying to a skin surface a composition comprising silicone and a particulate.

7. A method of increasing lubricity between two skin surfaces comprising applying a composition comprising silicone and a particulate to both said skin surfaces.
